(19) United States
(12) Patent Application Publication
(10) Pub. No.: US 2025/0057450 A1
(43) Pub. Date: Feb. 20, 2025

(54) Intelligent deformable microneedle and manufacturing method therefor

(71) Applicant: Peking University, Beijing (CN)

(72) Inventor: Yue CUI, Beijing (CN)

(73) Assignee: Peking University, Beijing (CN)

(21) Appl. No.: 18/796,282

(22) Filed: Aug. 6, 2024

(30) Foreign Application Priority Data
Aug. 15, 2023 (CN) 202311022073.6

(51) Int. Cl. A61B 5/1486; A61B 5/00; A61B 5/145; B33Y 80/00

(52) U.S. Cl. CPC A61B 5/14865; A61B 5/14532; A61B 5/685; A61B 2562/12; B33Y 80/00

(57) ABSTRACT

An intelligent deformable microneedle includes a supporting seat; a counter electrode provided above the supporting seat; an elastic object with a compressed state and a natural state, where a working electrode is provided on an outer surface of the elastic object, and a specific enzyme that can react with an analyte to be detected is provided on the working electrode; a soluble needle-shaped body fixed on the supporting seat, where the soluble needle-shaped body completely wraps the counter electrode and the elastic object from the outside, and the soluble needle-shaped body has an inner cavity structure that enables the elastic object to be in the compressed state. The microneedle is internally provided with the elastic object, after penetrating into skin, the soluble needle-shaped body is dissolved, the elastic object inside is exposed, length becomes longer, and the working electrode of an electrochemical sensor is attached to the elastic object.

### CROSS REFERENCE TO THE RELATED APPLICATIONS

This application is based upon and claims priority to Chinese Patent Application No. 202311022073.6, filed on Aug. 15, 2023, the entire contents of which are incorporated herein by reference.

### TECHNICAL FIELD

The present invention belongs to the technical field of microneedles, in particular to an intelligent deformable microneedle and a manufacturing method therefor.

### BACKGROUND

Combining microneedles and biomaterials to detect the concentration of corresponding components has been widely used in some treatments. For example, glucose oxidase is set on the microneedles, and then the microneedles penetrate the stratum corneum of the skin to reach the dermis, so as to complete the detection of the glucose concentration in the body of diabetic patients.

Most of the existing microneedles are in the shape of needles, and the microneedles are short, which makes the microneedles into the dermis short, for example, when detecting the concentration of glucose, the detection structure is not accurate enough.

Therefore, how to provide a microneedle that can solve the above defects has become an urgent technical problem for those skilled in the art.

### SUMMARY

Aiming at the technical problem in the prior art that a microneedle has poor accuracy in detecting concentration of a substance to be detected, the present invention further provides an intelligent deformable microneedle with an elastic object, so as to improve detection accuracy.

The technical solution used for solving the technical problem of the present invention is as follows:
The intelligent deformable microneedle includes: a supporting seat;
a counter electrode provided above the supporting seat and provided in a fixed state;
an elastic object with a compressed state and a natural state, the elastic object is provided above the supporting seat and provided in a fixed state, one end of the elastic object close to the supporting seat is a fixed end of the clastic object, and one end thereof far away from the supporting seat is a free end of the elastic object; when the free end is close to the fixed end, the clastic object is in the compressed state; and an outer surface of the elastic object is provided with a working electrode, the working electrode is electrically insulated from the counter electrode, and a specific enzyme which can react with an analyte to be detected is provided on the working electrode; and
a soluble needle-shaped body, which is fixed on the supporting seat with a needle tip thereof far away from the supporting seat, the soluble needle-shaped body completely wraps the counter electrode and the elastic object from the outside, and the soluble needle-shaped body has an inner cavity structure that enables the elastic object to be in the compressed state.

Preferably, the supporting seat is provided with a bearing surface, the elastic object extends along a direction perpendicular to the bearing surface, one end of the elastic object connected to the bearing surface is the fixed end, and the elastic object is provided in the fixed state; and when the elastic object is in the compressed state, a bottom end of the soluble needle-shaped body is fixedly provided on the bearing surface, the needle tip thereof is oriented in the direction perpendicular to the bearing surface, the inner cavity structure of the soluble needle-shaped body is a cavity, the counter electrode and the elastic object are located in the cavity, and an area surrounded by an inner side wall of the soluble needle-shaped body close to the needle tip thereof is less than a cross-sectional area of the free end.

Preferably, the intelligent deformable microneedle further includes a reference/counter electrode which is provided corresponding to the working electrode and the counter electrode, and a three-electrode system is formed.

Preferably, the clastic object is a spring.

Preferably, the intelligent deformable microneedle further includes a supporting body fixedly provided on the bearing surface, the counter electrode and the reference/counter electrode are provided on the supporting body, and the supporting body is provided in a fixed state.

Preferably, the supporting body is a second needle-shaped body which is provided in a solid shape, a bottom end of the second needle-shaped body is fixedly provided on the bearing surface, a needle tip thereof is oriented in the direction perpendicular to the bearing surface, and the second needle-shaped body is located in an inner space of the spring.

Preferably, the inner cavity structure corresponds to the spring in the compressed state and a structure of the second needle-shaped body.

Preferably, the supporting seat is in a cuboid shape.

Preferably, the soluble needle-shaped body is a water-soluble needle-shaped body.

Preferably, the specific enzyme is provided on the working electrode close to the free end of the elastic object.

The technical effects of the present invention are specifically as follows:

The microneedle is internally provided with the elastic object, after penetrating into skin, the soluble needle-shaped body is dissolved, the elastic object inside is exposed, its length becomes longer, and the working electrode of an electrochemical sensor is on the elastic object. In this way, although the length of the pierced microneedle is very short, the obtained working electrode may go deep into the skin and get accurate detection of a corresponding substance.

Preferably, the spring is selected as the elastic object, and is likely to deform.

A manufacturing method for an intelligent deformable microneedle is further provided by the present invention and includes: providing a supporting seat;
arranging a counter electrode provided in a fixed state above the supporting seat;
providing an elastic object with a compressed state and a natural state, arranging the elastic object above the supporting seat in a fixed state, making one end of the elastic object close to the supporting seat be a fixed end of the elastic object, making one end thereof far away from the supporting seat be a free end of the elastic object, when the free end is close to the fixed end, making the elastic object be in the compressed state, providing a working electrode on an outer surface of the elastic object, electrically insulating the working electrode from the counter electrode, and providing a specific enzyme which can react with an analyte to be detected on the working electrode; and
providing a soluble needle-shaped body, fixing the soluble needle-shaped body on the supporting seat with a needle tip thereof far away from the supporting seat, completely wrapping the counter electrode and the elastic object from the outside by the soluble needle-shaped body, and making the soluble needle-shaped body have an inner cavity structure that enables the elastic object to be in the compressed state.

Preferably, the manufacturing method for an intelligent deformable microneedle includes:
providing the supporting seat with a bearing surface;
fixedly providing the elastic object on the bearing surface, making the elastic object extend along a direction perpendicular to the bearing surface, making one end of the elastic object connected to the bearing surface be the fixed end, and making the other end be the free end;
providing the working electrode at the position of the free end of the elastic object;
providing the specific enzyme on the working electrode close the free end of the elastic object; and providing the counter electrode in the fixed state above the bearing surface; and
providing the soluble needle-shaped body with a cavity inside, fixedly providing a bottom end of the soluble needle-shaped body on the bearing surface, making the needle tip thereof be oriented in the direction perpendicular to the bearing surface, wrapping the counter electrode and the working electrode by the soluble needle-shaped body, and extruding the free end of the elastic object by the soluble needle-shaped body to compress the free end.

Preferably, the steps of providing the supporting seat with a bearing surface; fixedly providing the elastic object on the bearing surface, making the elastic object extend along a direction perpendicular to the bearing surface, making one end of the elastic object connected to the bearing surface be the fixed end, and making the other end be the free end; providing the working electrode at the position of the free end of the elastic object; providing the specific enzyme on the working electrode close the free end of the elastic object; and providing the counter electrode in the fixed state above the bearing surface” include:
designing the following model by using 3D modeling software: designing a cuboid as the supporting seat, designing a second needle-shaped body provided in solid on a surface of the cuboid, fixedly providing a bottom end of the second needle-shaped body on the surface of the cuboid, and making a needle tip thereof be oriented in the direction perpendicular to the bearing surface;
performing manufacturing by using a 3D printer according to the model, and then forming the counter electrode on a surface of the second needle-shaped body;
providing a spring as the elastic object, sleeving the spring outside the second needle-shaped body, fixedly providing the second needle-shaped body on the surface of the cuboid, making the spring extend and be provided along a direction perpendicular to the surface, making one end of the spring connected to the surface be the fixed end, making the other end be the free end, making the working electrode on the spring be not electrically connected to the counter electrode, and making the working electrode and the counter electrode be externally connected separately by connecting paste which is not electrically connected; and
subsequently, immobilizing the specific enzyme on the working electrode.

Preferably, the manufacturing method for an intelligent deformable microneedle further includes forming a reference/counter electrode on the surface of the second needle-shaped body, the reference/counter electrode is formed by coating silver/silver chloride slurry, and the working electrode and the counter electrode are formed by coating carbon slurry or gold slurry or platinum slurry or carbon-Prussian Blue composite slurry or gold-Prussian blue composite slurry or platinum-Prussian blue composite slurry.

Preferably, the manufacturing method for an intelligent deformable microneedle further includes forming a reference/counter electrode on the surface of the second needle-shaped body, which is formed by the following steps: evaporating or sputtering chromium or titanium as an adhesion layer, evaporating or sputtering thereon to obtain a gold or platinum electrode, evaporating or sputtering a silver film to obtain a silver electrode, then soaking the silver electrode in ferric chloride to become a silver/silver chloride electrode, and forming the working electrode by the following steps: evaporating or sputtering chromium or titanium as an adhesion layer, evaporating or sputtering thereon to obtain a gold or platinum electrode, and then electroplating Prussian blue thereon.

Preferably, glucose oxidase is provided as the specific enzyme as follows: dissolving glucose oxidase in a phosphate buffer solution to prepare a solution with a concentration of 10 U/μL, uniformly mixing the solution with a 0.5% glutaraldehyde solution at a volume ratio of 1:1, dropping a mixture onto the working electrode, drying at 4° C. for 24 h, and then washing off unimmobilized glucose oxidase with the phosphate buffer solution.

Preferably, the step of “providing a soluble needle-shaped body, fixing the soluble needle-shaped body on the supporting seat with a needle tip thereof far away from the supporting seat, completely wrapping the counter electrode and the elastic object from the outside by the soluble needle-shaped body, and making the soluble needle-shaped body have an inner cavity structure that enables the elastic object to be in the compressed state” includes:
the step uses a soft lithography method as follows:
firstly, printing and manufacturing a first mold with a 3D printer, the first mold including a cuboid-shaped outer shell provided in an opening, a needle-shaped body model protruding from a center of an inner bottom surface of the outer shell along a direction perpendicular to the inner bottom surface, making a needle tip of the needle-shaped body model be oriented in the direction perpendicular to the inner bottom surface, making the needle-shaped body model be higher than the second needle-shaped body, and making an area surrounded by an outer side wall close to the needle tip thereof be smaller than a cross-sectional area of the free end of the spring;
making a polydimethylsiloxane (PDMS) concave mold:mixing PDMS with a curing agent according to a mass ratio of 10:1, fully performing stirring in the middle to generate a large number of bubbles, then performing vacuumizing for 30 min to remove all bubbles, then pouring the obtained mixed liquid into the first mold, picking off bubbles generated in the middle with a needle, then performing drying in an oven under blowing condition of 65° C. for 4 h to obtain the PDMS concave mold corresponding to a needle-shaped concave mold of the needle-shaped body model; and the needle-shaped concave mold is higher than the second needle-shaped body, and an area surrounded by an outer side wall close to a needle tip of the needle-shaped concave mold is smaller than the cross-sectional area of the free end of the spring; and
placing a soluble material in a liquid state in the needle-shaped concave mold, covering the previously made supporting seat on a surface of the PDMS concave mold with the needle-shaped concave mold, making the free end of the spring extend into the needle-shaped concave mold and be compressed, after drying, separating the supporting seat from the PDMS concave mold, and fixedly connecting the surface of the supporting seat to the soluble needle-shaped body formed by the soluble material.

Preferably, the soluble material is a polyvinylpyrrolidone-polyvinyl alcohol (PVP-PVA) composite material, and a manufacturing method is as follows: weighing 0.6 g of PVA and 3.0 g of PVP, dissolving the PVA and the PVP in 20 mL of deionized water, heating the PVA and the PVP at 95° C. for 3 h to completely dissolve the PVA and the PVP, and then cooling the dissolved PVA and PVP to room temperature.

### BRIEF DESCRIPTION OF THE DRAWINGS

In order to describe the examples of the present application or the technical solutions in the prior art clearer, and the accompanying drawings required by the examples or description of the prior art are briefly described below. Obviously, the accompanying drawings in the following description show merely some examples described in the present application, and those of ordinary skill in the art would also be able to derive other accompanying drawings from these accompanying drawings without creative efforts.

FIG. 1 is a schematic structural diagram of a specific embodiment of an intelligent deformable microneedle provided by the present invention in a natural state.

FIG. 2 is a schematic diagram of a specific deformation process of the microneedle.

FIG. 3 is a schematic diagram showing measurement of specific sizes of the microneedle in a top view.

FIG. 4 is a schematic diagram showing the measurement of the specific sizes of the microneedle in side view.

FIG. 5 is a side view of a needle tip portion of a dissolved microneedle.

FIG. 6 is schematic diagram showing the measurement of specific sizes of the dissolved microneedle in side view.

FIG. 7 is a CV curve of hydrogen peroxide detected by the microneedle.

FIG. 8 is a graph showing the relationship between the scanning rates of hydrogen peroxide detected by the microneedle and the peak oxidation current.

FIG. 9 is an i-t curve of hydrogen peroxide detected by the microneedle at −0.1 V.

FIG. 10 is a calibration curve of hydrogen peroxide detected by the microneedle at −0.1 V.

FIG. 11 is a CV curve of glucose detected by the microneedle.

FIG. 12 is a graph showing the relationship between the scanning rates of glucose detected by the microneedle and the peak oxidation current.

FIG. 13 is an i-t curve of glucose detected by the microneedle at −0.1 V.

FIG. 14 is a calibration curve of glucose detected by the microneedle at −0.1 V.

FIG. 15 is an i-t curve of glucose detected by the microneedle at −0.1 V and different temperatures.

FIG. 16 is a graph showing the relationship between the current of glucose detected by the microneedle at −0.1 V and the temperature from 22° C. to 52° C.

FIG. 17 is an i-t curve of creatinine, uric acid and glucose detected by the microneedle at −0.1 V.

FIG. 18 is an i-t curve showing successive drops of creatinine, uric acid and glucose on the microneedle.

FIG. 19 is a graph showing the change of current values of repeated determination of glucose.

FIG. 20 shows the steps of providing the supporting seat with a bearing surface; fixedly providing an elastic object on a bearing surface, making the elastic object extend along a direction perpendicular to the bearing surface, making one end of the elastic object connected to the bearing surface be a fixed end, and making the other end be a free end; providing a working electrode at the position of the free end of the elastic object; providing a specific enzyme on the working electrode close the free end of the elastic object; and providing a counter electrode in a fixed state above the bearing surface” are a flow diagram.

FIG. 21 shows the step of “providing a soluble needle-shaped body, fixing the soluble needle-shaped body on a supporting seat with a needle tip thereof far away from the supporting seat, completely wrapping the counter electrode and the elastic object from the outside by the soluble needle-shaped body, and making the soluble needle-shaped body have an inner cavity structure that enables the elastic object to be in a compressed state” is a flow diagram.

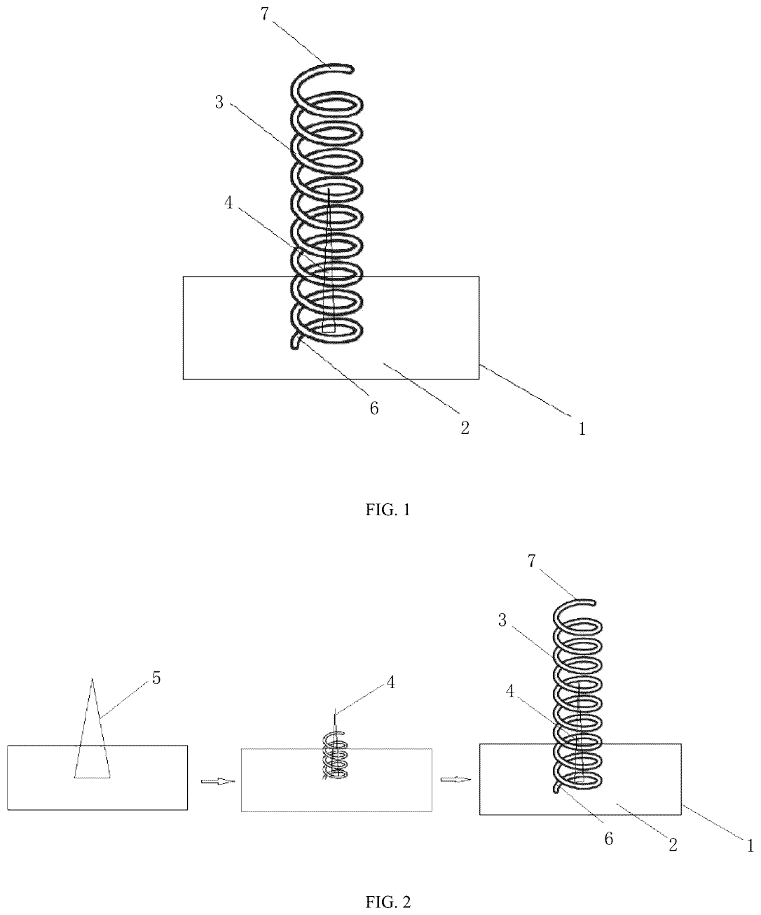

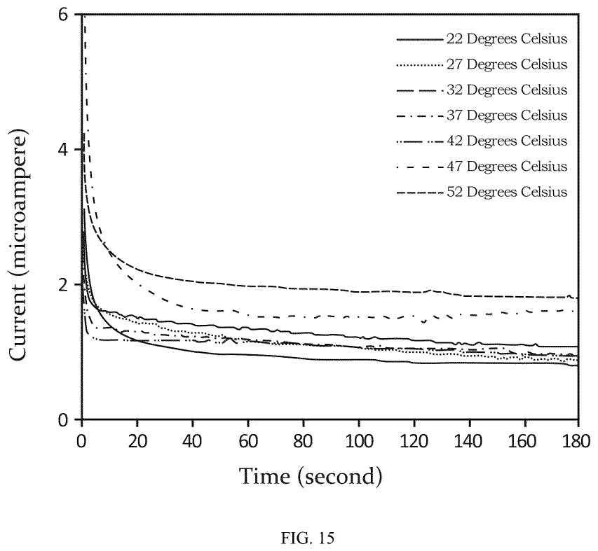

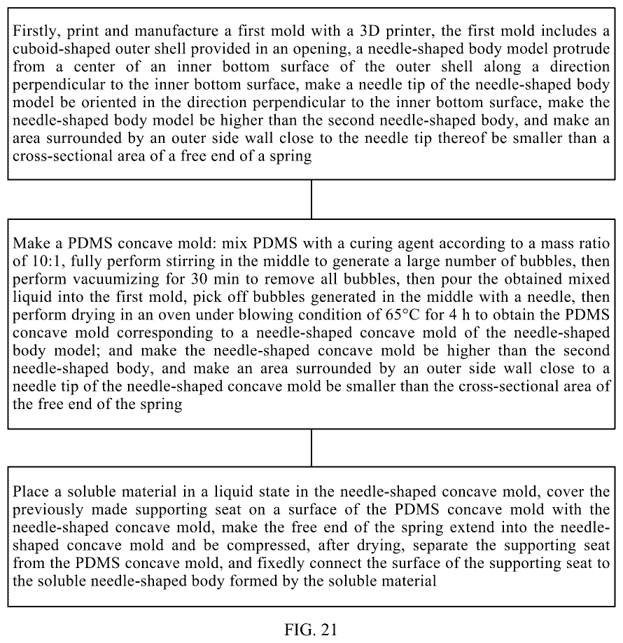

Reference numerals in FIGS. 1-21 as follows:
1 supporting seat, 2 bearing surface, 3 spring, 4 second needle-shaped body, 5 soluble needle-shaped body, 6 fixed end, 7 free end.

### DETAILED DESCRIPTION OF THE EMBODIMENTS

In order to make the technical solution of the present invention better understood by those skilled in the art, the present invention will be further described in detail bellow with reference to accompanying drawings and specific embodiments.

In a specific embodiment, an intelligent deformable microneedle includes a supporting seat 1 in a cuboid shape, the supporting seat is provided with a bearing surface 2, a spring 3 is used as an elastic object extending in a direction perpendicular to the bearing surface 2, one end of the spring 3 connected to the bearing surface 2 is a fixed end 6, and the other end is a free end 7; in a natural state, a second needle-shaped body 4 fixed to the bearing surface 2 is provided in an inner space of the spring 3, a working electrode is provided on the spring 3, and the second needle-shaped body 4 is provided with a reference electrode/counter electrode; and the intelligent deformable microneedle also includes a soluble needle-shaped body 5, which is fixed on the supporting seat 1 with a needle tip thereof far away from the supporting seat 1, the soluble needle-shaped body 5 completely wraps the spring 3 from the outside, and the soluble needle-shaped body 5 has an inner cavity structure that enables the spring 3 to be in a compressed state.

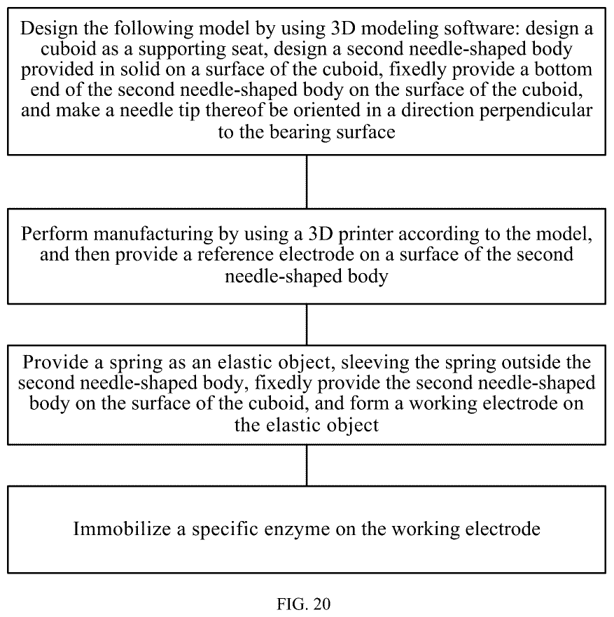

As for the inner cavity structure, in a specific embodiment, the inner cavity structure corresponds to the spring 3 in the compressed state and a structure of the second needle-shaped body 4; and in the specific embodiment, the soluble needle-shaped body 5 is formed by curing a soluble material in a liquid state, with no gap therein, that is, apart from the spring 3 in the compressed state and the second needle-shaped body 4, the rest are all soluble materials in a liquid state. Specifically, with reference to FIG. 20, the soluble needle-shaped body is formed by the following method:

firstly, printing and manufacturing a first mold with a 3D printer, the first mold including a cuboid-shaped outer shell provided in an opening, a needle-shaped body model protruding from a center of an inner bottom surface of the outer shell along a direction perpendicular to the inner bottom surface, making a needle tip of the needle-shaped body model be oriented in the direction perpendicular to the inner bottom surface, making the needle-shaped body model be higher than the second needle-shaped body 4, and making an area surrounded by an outer side wall close to the needle tip thereof be smaller than a cross-sectional area of the free end of the spring 3;

making a PDMS concave mold:mixing PDMS with a curing agent according to a mass ratio of 10:1, fully performing stirring in the middle to generate a large number of bubbles, then performing vacuumizing for 30 min to remove all bubbles, then pouring the obtained mixed liquid into the first mold, picking off bubbles generated in the middle with a needle, then performing drying in an oven under blowing condition of 65° C. for 4 h to obtain the PDMS concave mold corresponding to a needle-shaped concave mold of the needle-shaped body model; the needle-shaped concave mold is higher than the second needle-shaped body 4, and an area surrounded by an outer side wall close to a needle tip of the needle-shaped concave mold is smaller than the cross-sectional area of the free end of the spring 3; and placing a soluble material in a liquid state in the needle-shaped concave mold, covering the previously made supporting seat (the supporting seat 1 in a cuboid shape and with the spring 3 and the second needle-shaped body 4) on a surface of the PDMS concave mold with the needle-shaped concave mold, making the free end of the spring 3 extend into the needle-shaped concave mold and be compressed, after drying, separating the supporting seat 1 from the PDMS concave mold, and fixedly connecting the surface of the supporting seat 1 to the soluble needle-shaped body formed by the soluble material 5.

As for the inner cavity structure, in a specific embodiment, the inner cavity structure is a cavity, the counter electrode and the spring 3 are located in the cavity, and an area surrounded by an inner side wall of the soluble needle-shaped body 5 close to the needle tip thereof is less than a cross-sectional area of the free end.

For providing of electrodes, one method is:

evaporating or sputtering metal chromium (Cr) or titanium (Ti) on all electrodes as adhesion layers, obtaining gold or platinum electrodes by evaporation or sputtering, and then evaporating Prussian blue on the working electrode. On the reference/counter electrode, evaporating or sputtering a silver film to obtain a silver electrode, and then soaking the silver electrode in ferric chloride to become a silver/silver chloride electrode.

Another method is:

making slurry used for the working electrode and the counter electrode be generally carbon slurry or gold slurry or platinum slurry or carbon-Prussian blue composite slurry or gold-Prussian blue composite slurry or platinum-Prussian blue composite slurry. Silver/silver chloride slurry is used on the reference/counter electrode.

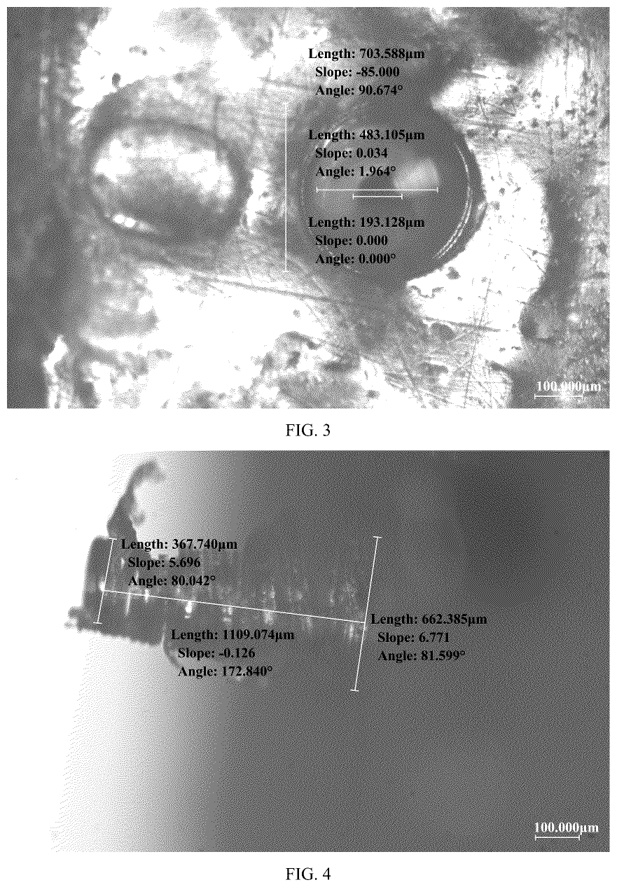

The spring 3 is fixed in the intelligent deformable microneedle in the compressed state. In the actual work, the soluble needle-shaped body of the intelligent deformable microneedle dissolves, the spring 3 is no longer forced to extend, and length of the spring 3 is longer than that of the original intelligent deformable microneedle (see FIG. 1 for the state diagram), such that the purpose of extending the microneedle is achieved in the whole process of dissolution and deformation. The specific deformation process is shown in FIG. 2.

Aiming to characterize the size of the intelligent deformable microneedle according to a specific embodiment of the present application, in order to display specific size information more clearly and intuitively, an optical microscope is additionally used to characterize the size of the obtained intelligent deformable microneedle:

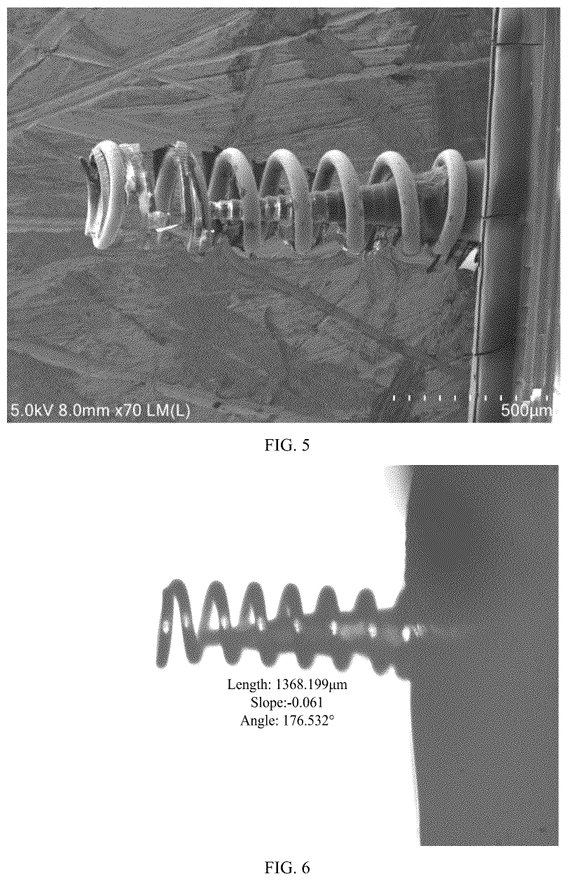

As shown in FIG. 3, it can be seen that sizes of the actually manufactured microneedle (bottom diameter of 703.6 μm, spring 3 with a diameter of 483.1 μm, second needle-shaped body 4 with a bottom diameter of 193.1 μm) are consistent with designed sizes (bottom diameter of 700 μm, spring with a diameter of 450 μm, inner microneedle with a bottom diameter of 200 μm).

As shown in FIG. 4, it can be seen that the size of the actually manufactured microneedle (bottom diameter of 662.4 μm, top diameter of 367.7 μm, height of 1109.1 μm) is consistent with the designed size (bottom diameter of 700 μm, top diameter of 200 μm, height of 1100 μm). The only big difference is the top diameter, which can be caused by excessive stress that compresses the microneedle.

Characterization of the dissolution and deformation performances of the intelligent deformable microneedle;

The obtained microneedle is completely immersed in deionized water and observed every 2 min, and the microneedle is completely dissolved in about 10 min.

Subsequently, the microneedle is taken out of the water, after drying, a needle tip portion of the microneedle is observed under an SEM and an optical microscope, the changes of length thereof are emphatically compared, as shown in FIG. 5 and FIG. 6, it can be found that the length of the microneedle changes from 1109.1 μm before dissolution to 1368.2 μm, which is similar to the spring length of 1400 μm, and the total elongation reached 20% or above. What mentioned above shows that the intelligent deformable microneedle has a certain deformation ability, and an elongation function of the working electrode after dissolution can be realized.

A test of detecting hydrogen peroxide by using the intelligent deformable microneedle: 1. a cyclic voltammetry curve of hydrogen peroxide is detected, hydrogen peroxide was analyzed as a product of glucose oxidation reaction, and also an actual detection object of the microneedle, and therefore, hydrogen peroxide is selected to test the performance of the microneedle at first. The first step is to find an appropriate detection potential by cyclic voltammetry.

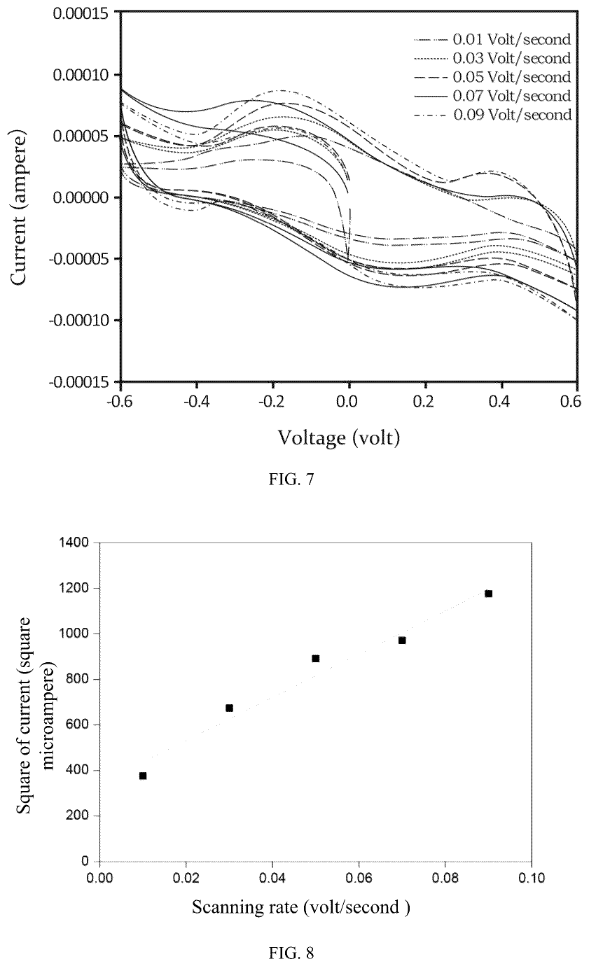

Hydrogen peroxide is diluted by phosphate buffer solution to produce hydrogen peroxide at a concentration of 10 mmol/L, the two electrodes of the microneedle (that is, the working electrode, the reference electrode/counter electrode) are connected to a power supply, and 10 μL of the solution to be tested is dropwise added on the needle tip portion of the microneedle (on the working electrode, which can cover the reference electrode/counter electrode, of course), a voltage range of −0.6 V to +0.6 V and a scanning rate of 0.01 V/s are selected, the diagram showing the relationship between the current and the voltage is measured, then, the scanning rate is changed to 0.03 V/s, 0.05 V/s, 0.07 V/s and 0.09 V/s, and the measurement results are shown in FIG. 7.

By observing FIG. 7, it can be found that the shapes of CV curves of detection of hydrogen peroxide at different rates are almost the same, but the peak values and the voltage of current peaks are different, by comparison, it is found that the current peak values decrease with the decrease of scanning rate, the corresponding voltage increases with the decrease of scanning rate, gradually approaching −0.1 V, which indicates that the optimal potential for time analysis of hydrogen peroxide current at constant voltage is around −0.1 V. Subsequent tests can be performed at −0.1 V. The current peak values at different scanning rates are recorded, as shown in the following table:

The current values for the detection of hydrogen peroxide with different scanning rates.

| Scanning rate (V/s) | 0.01 | 0.03 | 0.05 | 0.07 | 0.09 |
|---|---|---|---|---|---|
| Current (μA) | 19.39 | 25.96 | 29.85 | 31.16 | 34.30 |

It can be found that the magnitude of the current has a positive correlation with the scanning rate, which can be further tested by Randles-Sevcik equation:

$${i}_{p} = {kn}^{3} / {}^{2} {D}^{1} / {}^{2} {V}^{1} / {}^{2} Ac$$

In the formula, iₚ is peak current, n is the semi-reactive electron transfer number, D is a diffusion coefficient (cm²/s), v is voltage scanning speed (v/s), A is an area of the electrode (cm²), c is the concentration of a measured substance (mol/cm³), and k is a constant, and is 2.69×10⁵.

By incorporating the data into the proposed cooperative plot to obtain FIG. 8, it is found that the square of the current is approximately proportional to the scanning rate, indicating that the sensing process of hydrogen peroxide is primarily diffusion-controlled.

2. Analysis of a current-time curve for detecting hydrogen peroxide:

i-t-amperometry is used for measurement. The specific principle of an experiment is that a constant voltage is provided between the two electrodes of the microneedle as a stimulus, promoting the oxidation-reduction reactions of hydrogen peroxide at the electrodes to gain and lose electrons, which generates a current signal. The strength of the current signal allows the determination of the concentration of hydrogen peroxide produced.

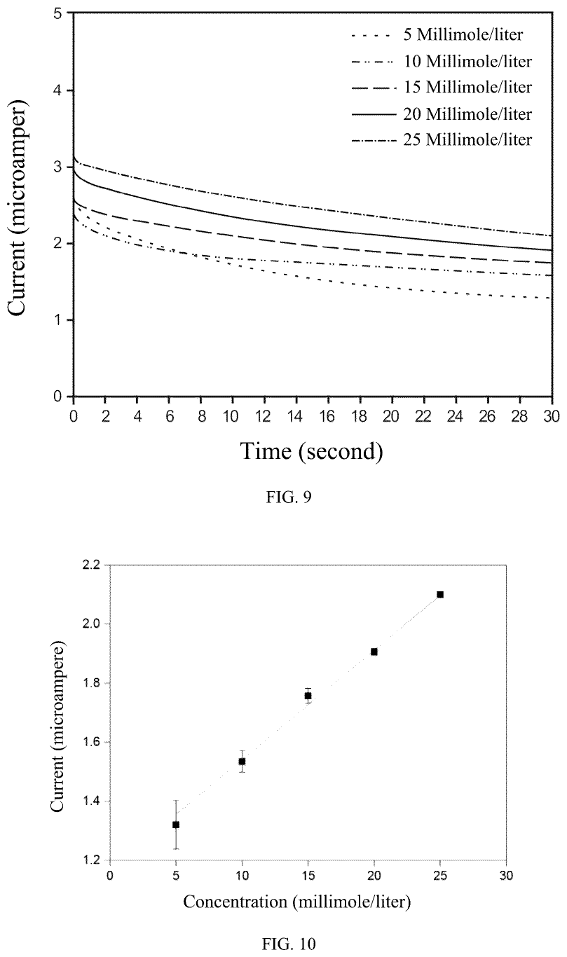

Hydrogen peroxide at a concentration of 30% (equivalent to a molar concentration of 9.9 mol/L) is dissolved in phosphate buffer solution to prepare a series of hydrogen peroxide solutions at concentrations of 5 mmol/L, 10 mmol/L, 15 mmol/L, 20 mmol/L and 25 mmol/L. The two electrodes of the microneedle are connected to the power supply, and 10 μL of the solution to be tested is added dropwise to the needle tip portion of the microneedle, the voltage of −0.1 V is selected to measure the graph of the current versus time, and the results of the measurements are shown in FIG. 9.

It can be found that the current generally shows a downward trend, the rate of decline gradually decreases, after 30 s, the current tends to be stable, the current values at various concentrations are recorded at the moment, the measurement is repeated for 3 times, and an average value is taken, as shown in the following table:

The current values for the detection of different concentrations of hydrogen peroxide.

| Concentration (mmol/L) | 5 | 10 | 15 | 20 | 25 |
|---|---|---|---|---|---|
| average value of i (μA) | 1.32 | 1.53 | 1.76 | 1.91 | 2.10 |

Subsequently, the obtained currents and the corresponding concentrations are fitted by the least square method, the fitting results are shown in FIG. 10, it can be found that the currents increase with the increase of hydrogen peroxide concentrations, and there is a good linear relationship between them, which indicates that the prepared microneedle can accurately determine the hydrogen peroxide concentrations, which also provides a basis for detection of glucose concentration.

The test of detecting glucose by using the intelligent deformable microneedle: 1. cyclic voltammetric curve analysis of glucose is detected, glucose is diluted with the phosphate buffer solution to prepare a glucose solution at a concentration of 30 mmol/L the two electrodes of the microneedle are connected to the power supply, 30 μL of the solution to be detected is dropwise added at the needle tip portion of the microneedle, the voltage range of −0.6 V-0.6 V, and the scanning rate of 0.01 V/s are selected, and the diagram showing the relationship between current and voltage is measured.

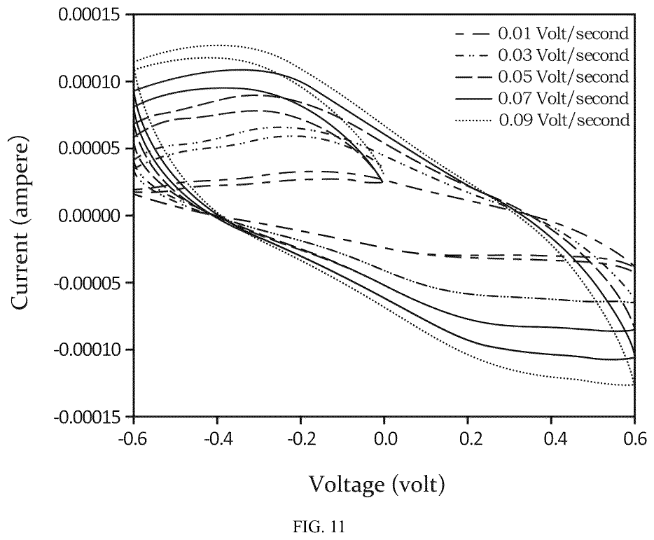

Subsequently, the scanning rate is changed to 0.03 V/s, 0.05 V/s, 0.07 V/s and 0.09 V/s, and the measurement results are shown in FIG. 11.

By observing FIG. 11, it can be found that the CV curves of glucose detected by the microneedle at different scanning rates are similar in shape, and the current peak values and the corresponding voltages change in the same trend with the decrease of scanning rates, the current peak values gradually decrease and the corresponding voltages gradually increase, in order to find a suitable reaction voltage, the current peak values and the corresponding voltages at the scanning rates are recorded, as shown in the following table:

Peak currents corresponding to voltage and scanning rates for glucose detection

| Scanning rate (V/s) | 0.01 | 0.03 | 0.05 | 0.07 | 0.09 |
|---|---|---|---|---|---|
| Current (μA) | 33.06 | 66.06 | 90.02 | 109.7 | 127.2 |
| Voltage (V) | −0.157 | −0.238 | −0.301 | −0.349 | −0.401 |

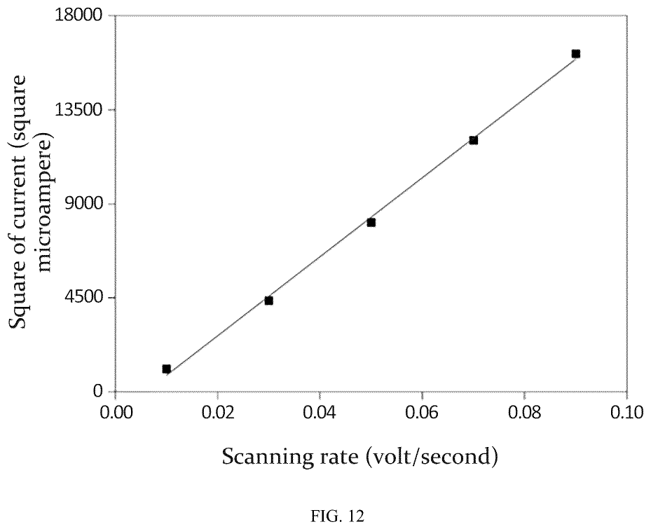

It can be found that with the decrease of the scanning rates, the voltage corresponding to the peak current gradually converges to −0.1 V, which indicates that the optimal potential for constant current time analysis of glucose is around −0.1 V, which is consistent with the conclusion obtained by cyclic voltammetry of hydrogen peroxide. Therefore, in the subsequent measurement of glucose concentration by chronoamperometry, the voltage of −0.1 V is used for the test. Further analysis and fitting of the square sum of current and scanning rates to obtain FIG. 12, it can be found that the square sum of current and scanning rate have a good linear relationship, which is consistent with the Randles-Savcik equation, indicating that the glucose sensing is mainly determined by diffusion, similar to hydrogen peroxide.

### 2. Analysis of a Current-Time Curve for Detecting Glucose

Same as the detection of hydrogen peroxide, the measurement is performed by chronoamperometry. The specific principle of detecting glucose in the experiment is that hydrogen peroxide is generated in the process of glucose oxidation by glucose oxidase on the microneedle, under a given excitation voltage, hydrogen peroxide is oxidized and reduced at the electrodes to gain and lose the electrons, a current signal is generated, the concentration of hydrogen peroxide can be determined by the strength of the current signal, and the concentration of glucose can be estimated by combining a coefficient ratio of glucose to hydrogen peroxide in the glucose oxidation reaction of 1:1.

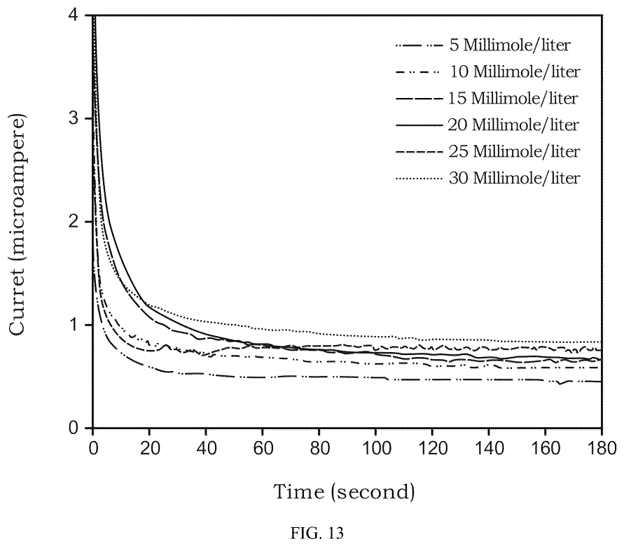

Glucose is dissolved in a phosphate buffer solution to prepare a series of glucose solutions having concentrations of 5 mmol/L, 10 mmol/L, 15 mmol/L, 20 mmol/L, 25 mmol/L and 30 mmol/L, the two electrodes of the microneedle are connected to the power supply, and 10 μL of the solution to be tested is added dropwise to the needle tip portion of the microneedle, the voltage of −0.1 V is selected, the diagram showing the relationship between current and time is measured, and the measurement results are shown in FIG. 13.

Since the redox of glucose takes a certain time, the test time is longer than that of hydrogen peroxide, by observing FIG. 13, it can be found that, similar to hydrogen peroxide, the current for detecting glucose also decreases with the increase of time, and the rate of decrease gradually decreases, and finally tends to be stable; and then, the current at 130 s where the current tends to be stable is selected, the current values at various concentrations are recorded, the measurement is repeated for 3 times, and the average value is taken, as shown in the following table:

### Current Values for Glucose Detection

| | Concentration (mmol/L) | | | | | |
|---|---|---|---|---|---|---|
| | 5 | 10 | 15 | 20 | 25 | 30 |
| average value | 0.4741 | 0.5992 | 0.6501 | 0.7116 | 0.7643 | 0.8519 |
| of i (μA) | | | | | | |

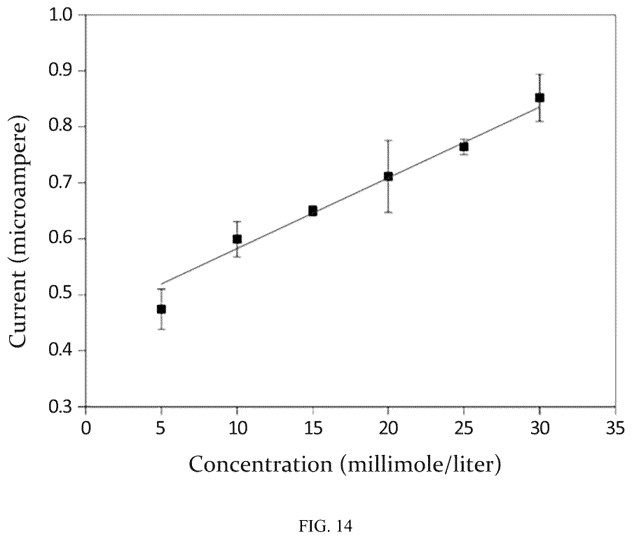

The current and the corresponding glucose concentrations are fitted, the fitting results are shown in FIG. 14, it can be seen that there is a good linear relationship between the magnitude of the current and the glucose concentration, which indicates that the prepared soluble intelligent microneedle can measure the glucose concentration well.

Different from the detection of hydrogen peroxide, the experiment should pay attention to one thing: because the enzyme-catalyzed reaction is affected by a contact area, the glucose solution should be dripped into the same area every time and the contact area of droplets with the microneedle is ensured as much as possible, and the concentration of glucose and the concentration of hydrogen peroxide obtained by enzyme-catalyzed oxidation is ensured as much as possible, such that the accuracy of experimental results is ensured.

Influence of different temperatures on the detection of glucose by the microneedle:

Human body temperature is generally 37° C., the working temperature of the microneedle is also around 37° C., therefore, the range of 37±15° C. is selected to detect the concentration of glucose with the microneedle, and the influence of temperature on the detection of glucose with the microneedle is explored.

A glucose solution at a concentration of 30 mmol/L is selected for measurement, the electrodes and the glucose solution are placed above a hot plate for heating, the two electrodes of the microneedle are connected to the power supply, 10 μL of the solution to be measured is dropwise added at the needle tip portion of the microneedle, a voltage of −0.1 V is selected, heating is started at 22° C., heating is stopped every 5° C., the temperature is kept stable for a period of time, and the i-t curve at 22° C. to 55° C. is measured. The results are shown in Table 15.

It can be found that the i-t curve of glucose detected by the microneedle at different temperatures is also a curve with decreasing rate, the current at 130 s where the current tends to be stable is still selected, and the current values at various concentrations are recorded, as shown in the following table:

Current values for glucose detection by the microneedle at different temperatures at 130 s.

| | Temperature (° C.) | | | | | | |
|---|---|---|---|---|---|---|---|
| | 22 | 27 | 32 | 37 | 42 | 47 | 52 |
| Current | 0.8519 | 0.9692 | 1.031 | 1.057 | 1.148 | 1.519 | 1.888 |
| (μA) | | | | | | | |

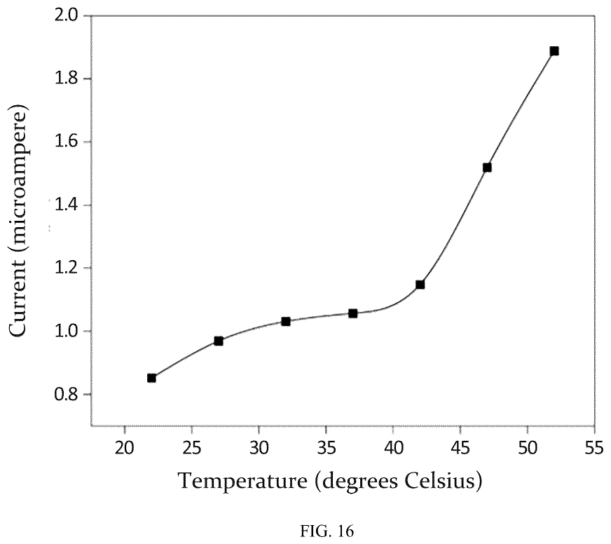

The temperature and the corresponding current are drawn to obtain FIG. 16, by observing FIG. 16, it can be found that with the temperature rising in an experimental temperature range (22° C. to 55° C.), the detected current value gradually increases, indicating that the concentration of hydrogen peroxide generated by oxidizing glucose by glucose oxidase increases, that is, the activity of glucose oxidase increases; and on the other hand, the rate of increase of glucose oxidase activity increases greatly from 40° C.

According to data, the working temperature of glucose oxidase is 30-60° C., and the most suitable working temperature is 50-55° C., which is consistent with experimental results.

Influence of different interfering substances on the detection of glucose by the microneedle:

Common interfering substances in blood glucose detection mainly include creatinine, uric acid, lactic acid, ascorbic acid, dopamine, etc. (see the table below), two methods of dropping separately and dropping continuously can be used to detect influence of interfering substances on blood glucose detection.

| Substance | Concentration |
|---|---|
| Creatinine | Male: 60-110 μmol/L |
| | Female: 45-90 μmol/L |
| Uric acid | Male: 149-416 μmol/L |
| | Female: 89-357 μmol/L |
| Lactic acid | Colorimetric determination: <2.4 mmol/L |
| Ascorbic acid | About 50 μmol/L |
| Dopamine | <1 μmol/L |

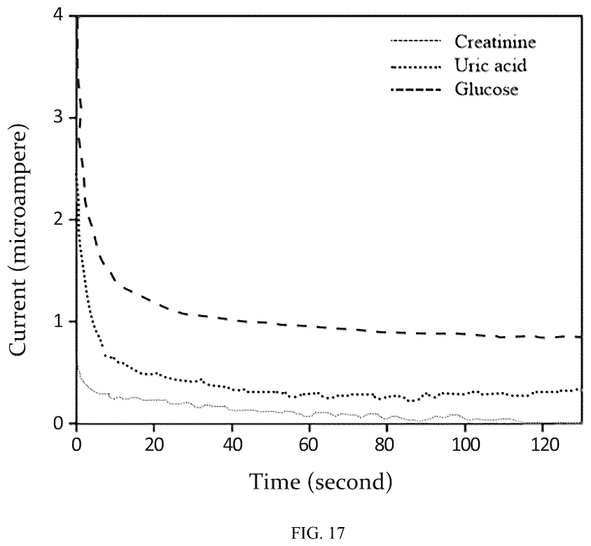

Creatinine and uric acid with high concentrations in blood are selected as the interfering substances for glucose detection, creatinine, uric acid and glucose are dissolved in the phosphate buffer solution to prepare solutions having concentrations of 30 mmol/L separately, and the solutions are detected by the method of dropping separately. The two electrodes of the microneedle are connected to the power supply, 10 μL of creatinine solution is dropwise added at the needle tip portion of the microneedle, the voltage of −0.1 V is selected, the diagram showing relationship between current and time, then, a pipette is used to suck the remaining solution, then a uric acid solution is dropwise added for measurement after drying, and finally, a change curve of current with time of the glucose solution is measured. Results are shown in FIG. 17.

By observing FIG. 17, it can be found that the current of glucose detected by the microneedle is much larger than that of detecting creatinine and uric acid, indicating that the microneedle has high selectivity for glucose.

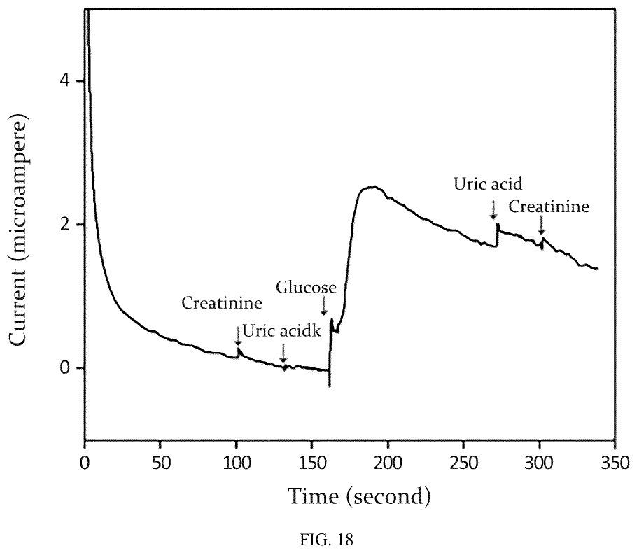

In order to further verify the selectivity of the microneedle in detecting glucose, the continuous dropping method can be used at a voltage of −0.1 V, 50 μL of phosphate buffer solution is added dropwise to the electrode, 5 μL of creatinine is added dropwise after the current stabilizes (about 100 s), 5 μL of uric acid is added dropwise after 30 s, 5 μL of glucose is added dropwise after 30 s, 5 μL of uric acid is added dropwise anew after the current stabilizes (about 270 s), and 5 μL of creatinine is added dropwise after 30 s. The results are shown in FIG. 18. Since diffusion takes a certain time, the current of glucose detection does not reach the peak at the moment of dropwise addition, and the larger change in current when uric acid an creatinine are added dropwise for the second time may be due to the fact that some glucose is still reacting.

After two experiments, it can be found that the response of the microneedle to glucose is obviously greater than its response to creatinine and uric acid. This indicates that microneedle has high specificity for glucose and can effectively avoid the influence of other interfering substances.

### Stability Test for Detecting Glucose:

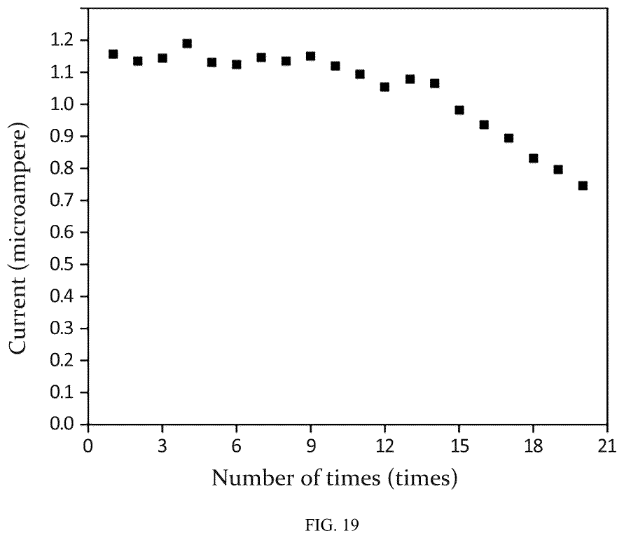

In order to detect the stability of the microneedle, repeated experiments are selected, the glucose solution at a concentration of 30 mmol/L is determined several times, and the current value at 130 s is recorded. Before each repetition, it is ensured that the microneedle is fully dried after the last test, the glucose solution is dropwise added on the same position of the microneedle every time to ensure the same contact area as far as possible, after the experiment is repeated for 20 times, the obtained current values are drawn to obtain FIG. 19.

It can be found that within 15 tests, the deviation of the current value of glucose detected by the microneedle is within 10%, which indicates that the prepared microneedle can measure the glucose concentration well and can still ensure a certain accuracy after repeated measurements.

The microneedle prepared by the present application can be completely dissolved in water within 10 min, and can complete the stretching deformation of the spring 3, with the elongation reaching 20% or more. An actual detection effect of hydrogen peroxide and glucose by the working electrode of the microneedle is tested. Cyclic voltammetry (CV) is used to test the suitable voltage of the microneedle for glucose sensing, i-t-amperometry is used to characterize the concentration of hydrogen peroxide and glucose detected by the working electrode of the microneedle, further, the influence of temperature and other interfering substances on the detection of glucose by the microneedle is also studied, and the current attenuation of repeated detection of glucose by the microneedle is also characterized. The results show that the microneedle can detect the glucose concentration accurately, the detection sensitivity changes with the temperature, the influence of the interfering substances such as creatinine and uric acid on glucose detection can be eliminated, and the microneedle has high detection accuracy within a certain number of detection times.

According to the manufacturing method used for an intelligent deformable microneedle, in a specific embodiment, the supporting seat 1 and the second needle-shaped body 4, which is fixedly connected thereto are manufactured by 3D printing. Then, the second needle-shaped body is coated with silver/silver chloride paste to serve as the reference electrode/counter electrode. Afterward, the spring 3 is sleeved outside the second needle-shaped body 4, and the working electrode is provided on the spring 3.

As shown in FIG. 20, the first mold is initially printed and manufactured with a 3D printer, the first mold includes the cuboid-shaped outer shell provided in the opening, the needle-shaped body model protruding from the center of the inner bottom surface of the outer shell along the direction perpendicular to the inner bottom surface, a needle tip of the needle-shaped body model is oriented in the direction perpendicular to the inner bottom surface, the needle-shaped body model is higher than the second needle-shaped body, and the area surrounded by the outer side wall close to the needle tip thereof is smaller than the cross-sectional area of the free end of the spring;
the PDMS concave mold is made: the PDMS is mixed with the curing agent according to the mass ratio of 10:1, stirring is fully performed in the middle to generate the large number of bubbles, then vacuumizing is performed for 30 min to remove all bubbles, then the obtained mixed liquid is poured into the first mold, the bubbles generated in the middle are picked off with the needle, then drying is performed in the oven under the blowing condition of 65° C. for 4 h to obtain the PDMS concave mold corresponding to the needle-shaped concave mold of the needle-shaped body model; the needle-shaped concave mold is higher than the second needle-shaped body, and the area surrounded by the outer side wall close to the needle tip of the needle-shaped concave mold is smaller than the cross-sectional area of the free end of the spring; and
a soluble liquid material is placed in the needle-shaped concave mold, the previously made supporting seat is covered on the surface of the PDMS concave mold with the needle-shaped concave mold, the free end of the spring extends into the needle-shaped concave mold and is compressed, after drying, the supporting seat is separated from the PDMS concave mold, and the surface of the supporting seat is fixedly connected to the soluble needle-shaped body formed by the soluble material.

It can be understood that the above embodiments are only exemplary embodiments used for explaining the principles of the present invention, but the present invention is not limited thereto. Those of ordinary skill in the art can make several modifications and improvements without departing from the spirit and the principles of the present invention, and all the modifications and improvements will also fall within the scope of protection of the present invention.

What is claimed is:

1. An intelligent deformable microneedle, comprising:
a supporting seat;
a counter electrode provided above the supporting seat and provided in a first fixed state;
an elastic object with a compressed state and a natural state, wherein the elastic object is provided above the supporting seat and provided in a second fixed state, a first end of the elastic object adjacent to the supporting seat is a fixed end of the elastic object, and a second end of the elastic object far away from the supporting seat is a free end of the elastic object; when the free end is adjacent to the fixed end, the elastic object is in the compressed state; and an outer surface of the elastic object is provided with a working electrode, the working electrode is electrically insulated from the counter electrode, and a specific enzyme configured to react with an analyte to be detected is provided on the working electrode; and
a soluble needle-shaped body, wherein the soluble needle-shaped body is fixed on the supporting seat, and a needle tip of the soluble needle-shaped body is far away from the supporting seat; the soluble needle-shaped body completely wraps the counter electrode and the elastic object from an outside, and the soluble needle-shaped body has an inner cavity structure configured to enable the elastic object to be in the compressed state.

2. The intelligent deformable microneedle according to claim 1, wherein the supporting seat is provided with a bearing surface, the elastic object extends along a direction perpendicular to the bearing surface, the fixed end of the elastic object is connected to the bearing surface, and the elastic object is provided in the second fixed state; and when the elastic object is in the compressed state, a bottom end of the soluble needle-shaped body is fixedly provided on the bearing surface, the needle tip of the soluble needle-shaped body is oriented in the direction perpendicular to the bearing surface, the inner cavity structure of the soluble needle-shaped body is a cavity, the counter electrode and the elastic object are located in the cavity, and an area surrounded by an inner side wall of the soluble needle-shaped body adjacent to the needle tip is less than a cross-sectional area of the free end.

3. The intelligent deformable microneedle according to claim 1, further comprising a reference electrode, wherein the reference electrode is provided corresponding to the working electrode and the counter electrode to form a three-electrode system.

4. The intelligent deformable microneedle according to claim 3, wherein the elastic object is a spring.

5. The intelligent deformable microneedle according to claim 4, further comprising a supporting body fixedly provided on the bearing surface, wherein the counter electrode and the reference electrode are provided on the supporting body, and the supporting body is provided in a third fixed state.

6. The intelligent deformable microneedle according to claim 5, wherein the supporting body is a second needle-shaped body and is provided in a solid shape, a bottom end of the second needle-shaped body is fixedly provided on the bearing surface, a needle tip of the second needle-shaped body is oriented in the direction perpendicular to the bearing surface, and the second needle-shaped body is located in an inner space of the spring.

7. The intelligent deformable microneedle according to claim 1, wherein the inner cavity structure corresponds to the spring in the compressed state and a structure of the second needle-shaped body.

8. The intelligent deformable microneedle according to claim 1, wherein the supporting seat is in a cuboid shape.

9. The intelligent deformable microneedle according to claim 1, wherein the soluble needle-shaped body is a water-soluble needle-shaped body.

10. The intelligent deformable microneedle according to claim 1, wherein the specific enzyme is provided on the working electrode adjacent to the free end of the elastic object.

11. A manufacturing method for an intelligent deformable microneedle, comprising:
providing a supporting seat;
arranging a counter electrode provided in a first fixed state above the supporting seat;
providing an elastic object with a compressed state and a natural state, arranging the elastic object above the supporting seat in a second fixed state, making a first end of the elastic object adjacent to the supporting seat be a fixed end of the elastic object, making a second end of the elastic object far away from the supporting seat be a free end of the elastic object, when the free end is adjacent to the fixed end, making the elastic object be in the compressed state, providing a working electrode on an outer surface of the elastic object, electrically insulating the working electrode from the counter electrode, and providing a specific enzyme configured to react with an analyte to be detected on the working electrode; and
providing a soluble needle-shaped body, fixing the soluble needle-shaped body on the supporting seat with a needle tip of the soluble needle-shaped body far away from the supporting seat, completely wrapping the counter electrode and the elastic object from an outside by the soluble needle-shaped body, and making the soluble needle-shaped body have an inner cavity structure configured to enable the elastic object to be in the compressed state.

12. The manufacturing method according to claim 11, comprising:
providing the supporting seat with a bearing surface;
fixedly providing the elastic object on the bearing surface, making the elastic object extend along a direction perpendicular to the bearing surface, making the first end of the elastic object connected to the bearing surface be the fixed end, and making the second end of the elastic object be the free end;
providing the working electrode at a position of the free end of the elastic object;
providing the specific enzyme on the working electrode adjacent to the free end of the clastic object;
providing the counter electrode in the first fixed state above the bearing surface; and
providing the soluble needle-shaped body with a cavity inside, fixedly providing a bottom end of the soluble needle-shaped body on the bearing surface, making the needle tip of the soluble needle-shaped body be oriented in the direction perpendicular to the bearing surface, wrapping the counter electrode and the working electrode by the soluble needle-shaped body, and extruding the free end of the elastic object by the soluble needle-shaped body to compress the free end.

13. The manufacturing method according to claim 12, wherein the steps of providing the supporting seat with the bearing surface; fixedly providing the elastic object on the bearing surface, making the elastic object extend along the direction perpendicular to the bearing surface, making the first end of the elastic object connected to the bearing surface be the fixed end, and making the second end of the elastic object be the free end; providing the working electrode at the position of the free end of the elastic object; providing the specific enzyme on the working electrode adjacent to the free end of the elastic object; and providing the counter electrode in the first fixed state above the bearing surface comprise:
designing a model as follows by using 3D modeling software: designing a cuboid as the supporting seat, designing a second needle-shaped body provided in solid on a surface of the cuboid, fixedly providing a bottom end of the second needle-shaped body on the surface of the cuboid, and making a needle tip of the second needle-shaped body be oriented in the direction perpendicular to the bearing surface;
performing manufacturing by using a 3D printer according to the model, and then providing the counter electrode on a surface of the second needle-shaped body;
providing a spring as the elastic object, sleeving the spring outside the second needle-shaped body, fixedly providing the second needle-shaped body on the surface of the cuboid, making the spring extend and be provided along a direction perpendicular to the surface, making a first end of the spring connected to the surface be the fixed end, making a second end of the spring be the free end, making the working electrode on the spring be not electrically connected to the counter electrode, and making the working electrode and the counter electrode be externally connected separately by connecting paste, wherein the connecting paste is not electrically connected; and
subsequently, fixing the specific enzyme on the working electrode.

14. The manufacturing method according to claim 13, further comprising forming a reference electrode on the surface of the second needle-shaped body, wherein the reference electrode is formed by coating silver/silver chloride slurry, and the working electrode and the counter electrode are formed by coating carbon slurry or gold slurry or platinum slurry or carbon-Prussian Blue composite slurry or gold-Prussian blue composite slurry or platinum-Prussian blue composite slurry.

15. The manufacturing method according to claim 13, further comprising: forming a reference electrode on the surface of the second needle-shaped body by the following steps: evaporating or sputtering chromium or titanium as a first adhesion layer, evaporating or sputtering on the first adhesion layer to obtain a first gold or platinum electrode, evaporating or sputtering a silver film to obtain a silver electrode, then soaking the silver electrode in ferric chloride to become a silver/silver chloride electrode; and
forming the working electrode by the following steps: evaporating or sputtering chromium or titanium as a second adhesion layer, evaporating or sputtering on the second adhesion layer to obtain a second gold or platinum electrode, and then electroplating Prussian blue thereon.

16. The manufacturing method according to claim 13, wherein
the step of providing glucose oxidase as the specific enzyme comprises: dissolving the glucose oxidase in a phosphate buffer solution to prepare a solution with a concentration of 10 U/μL, uniformly mixing the solution with a 0.5% glutaraldehyde solution at a volume ratio of 1:1 to obtain a mixture, dropping the mixture onto the working electrode, drying at 4° C. for 24 h, and then washing off un-immobilized glucose oxidase with the phosphate buffer solution.

17. The manufacturing method according to claim 11, wherein the step of providing the soluble needle-shaped body, fixing the soluble needle-shaped body on the supporting seat with the needle tip of the soluble needle-shaped body far away from the supporting seat, completely wrapping the counter electrode and the elastic object from the outside by the soluble needle-shaped body, and making the soluble needle-shaped body have the inner cavity structure configured to enable the elastic object to be in the compressed state comprises:
the step uses a soft lithography method as follows:
firstly, printing and manufacturing a first mold with a 3D printer, the first mold comprising a cuboid-shaped outer shell provided in an opening, a needle-shaped body model protruding from a center of an inner bottom surface of the outer shell along a direction perpendicular to the inner bottom surface, making a needle tip of the needle-shaped body model be oriented in the direction perpendicular to the inner bottom surface, making the needle-shaped body model be higher than a second needle-shaped body, and making an area surrounded by an outer side wall adjacent to the needle tip of the needle-shaped body model be smaller than a cross-sectional area of a free end of a spring;
making a polydimethylsiloxane (PDMS) concave mold:mixing PDMS with a curing agent according to a mass ratio of 10:1, fully performing stirring in a middle to generate a large number of bubbles, then performing vacuumizing for 30 min to remove all bubbles, then pouring an obtained mixed liquid into the first mold, picking off bubbles generated in the middle with a needle, then performing drying in an oven under blowing condition of 65° C. for 4 h to obtain the PDMS concave mold corresponding to a needle-shaped concave mold of the needle-shaped body model; wherein the needle-shaped concave mold is higher than the second needle-shaped body, and an area surrounded by an outer side wall adjacent to a needle tip of the needle-shaped concave mold is smaller than the cross-sectional area of the free end of the spring; and
placing a soluble material in a liquid state in the needle-shaped concave mold, covering the supporting seat on a surface of the PDMS concave mold with the needle-shaped concave mold, making the free end of the spring extend into the needle-shaped concave mold and be compressed, after drying, separating the supporting seat from the PDMS concave mold, and fixedly connecting a surface of the supporting seat to the soluble needle-shaped body formed by a soluble material.

18. The manufacturing method according to claim 17, wherein the soluble material is a polyvinylpyrrolidone-polyvinyl alcohol (PVP-PVA) composite material, and a manufacturing method is as follows:
weighing 0.6 g of PVA and 3.0 g of PVP, dissolving the PVA and the PVP in 20 mL deionized water, heating the PVA and the PVP at 95° C. for 3 h to completely dissolve the PVA and the PVP, and then cooling dissolved PVA and PVP to room temperature.

19. The intelligent deformable microneedle according to claim 2, further comprising a reference electrode, wherein the reference electrode is provided corresponding to the working electrode and the counter electrode to form a three-electrode system.

20. The intelligent deformable microneedle according to claim 19, wherein the elastic object is a spring.
